(19) United States
(12) Patent Application Publication
(10) Pub. No.: US 2009/0305315 A1
(43) Pub. Date: Dec. 10, 2009

(54) HUBBED DUAL CANNULA DEVICE FOR CLOSED CONTAINER SAMPLING SYSTEMS

(76) Inventors: Kent Raphael Gandola, San Diego, CA (US); David Elliott Kaplan, Carlsbad, CA (US); Rex O. Bare, Lake Forest, CA (US); Christopher Johann Johnson, San Diego, CA (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/480,476

(22) Filed: Jun. 8, 2009

Related U.S. Application Data
(60) Provisional application No. 61/060,076, filed on Jun. 9, 2008.

(51) Int. Cl. C12Q 1/56; C12M 1/26; C12Q 1/02

(52) U.S. Cl. 435/13; 435/309.1; 435/29

(57) ABSTRACT

The present invention provides a hubbed dual cannula device for extracting a fluid sample from a closed container. The device comprises a housing hub, two substantially parallel needles, a venting valve and a hydrophobic membrane, and may be used effectively to extract a fluid sample from a sealed container, such as a vacuum tube, without damaging the cellular components or activating platelets in the fluid sample. Also provided are methods for using the disclosed device to extract a fluid sample from a closed container and kits for measuring platelet aggregation using such a device.

### CROSS-REFERENCE TO RELATED APPLICATIONS

This application claims the benefit of U.S. Ser. No. 61/060,076, filed Jun. 9, 2008, which is incorporated herein by reference in its entirety.

### FIELD OF THE INVENTION

This invention generally relates to the field of diagnostic assays, particularly to container sampling systems, such as clinical chemistry analyzers, and more particularly to hubbed dual cannula devices for extracting a fluid sample from a closed container.

### BACKGROUND OF THE INVENTION

The ability to measure quantitatively a wide variety of physiologically active compounds, both naturally occurring and synthetic, has become of increasing importance, both as an adjunct to diagnosis and therapy. The medical industry has become increasingly dependent upon the ability to measure various entities in physiological fluids in order to be able to determine the health status of an individual, dosage level for drugs, use of illegal drugs, genomic sequences and the like. Thus, the capability of taking a physiological sample and rapidly analyzing for a particular component has made medical therapies more efficient and increasingly successful.

For the most part diagnostic assays of physiological fluids or biological samples for one or more analytes have required clinical laboratory determinations although there has been an increasing focus on being able to carry out assay determinations in the doctor's office and in the home. Numerous systems have been developed in efforts to try to address the various problems associated with analyses carried out in the clinical laboratory.

There is substantial interest in providing for protocols and devices which are simple, easy to manipulate, and reduce the opportunity for operator failure. The ideal situation would be collection of an unmeasured sample in a container, which is then sealed. Subsequently, the sample could then be introduced into an assay device without opening the sealed container and without the need for accurately measuring the sample. The device into which the sample is introduced provides for precise measurement of the sample to be analyzed, which is important in obtaining a quantitative result.

In many instances blood is a source of a sample to diagnose a patient's health or to monitor the efficacy of drugs that have been administered to the patient. Blood as a source for the determination of these parameters has many deficiencies when used directly or even when diluted with buffer. These deficiencies include: rapid coagulation, the presence of a large number of light absorbing and fluorescent substances, variations in composition, susceptibility to changes in relation to reagents used in assays, and variations in the presence or absence of oxygen. These properties complicate the use of blood as a sample for diagnostic purposes. Various techniques have been employed to avoid these problems, e.g., high dilution, addition of anticoagulants, separation of blood into plasma and its cellular components, and the like. During such manipulations great care must be taken to avoid lysis of red blood cells to avoid the release of hemoglobin, which can interfere with certain diagnostic assays. Despite the problems associated with the use of blood as the sample medium, in many instances, blood is the only source that provides the information of interest. Therefore, identifying ways of using whole blood, while diminishing the interference from its constituents, is highly desirable. There is, therefore, substantial interest in improving approaches for using and manipulating blood for diagnostic purposes.

One area of particular interest in analyses employing whole blood samples is the assessment of platelet function. The role of platelets in mammalian physiology is extraordinarily diverse, but their primary role is in promoting thrombus formation. In many situations, an evaluation of the ability of blood to clot is desired, a parameter that is frequently controlled by the ability of platelets to adhere and/or aggregate. Thus, one may wish to assess the adhesive functions of platelets. For example, one may wish to know whether to administer drugs that will block, or promote, clot formation, or one may need to detect deficiencies in platelet function prior to surgical procedures. In other instances one may be interested in evaluating the effectiveness of a platelet inhibitor that is being tested as a new drug or is being used as approved clinical treatment in a patient.

Platelets play a critical role in the maintenance of normal hemostasis. When exposed to a damaged blood vessel, platelets will adhere to exposed sub-endothelial matrix. Following the initial adhesion, various factors released or produced at the site of injury such as thrombin, ADP and collagen activate the platelets. Once platelets are activated, a conformational change occurs in the platelet glycoprotein GPIIb/IIIa receptor, allowing it to bind fibrinogen and/or von Willebrand factor. It is this binding of the multivalent fibrinogen and/or von Willebrand factor molecules by GPIIb/IIIa receptors on adjacent platelets that results in the recruitment of additional platelets to the site of injury and their aggregation to form a hemostatic plug or thrombus.

A rapid platelet function assay has been developed and is described in U.S. Pat. No. 5,763,199 (Coller), which is fully incorporated herein by reference. The assay determines glycoprotein GPIIb/IIIa receptor blockade in whole blood. Agglutination of small polymeric beads coated with a GPIIb/IIIa ligand such as fibrinogen results when the beads are contacted with whole blood containing platelets with activated GPIIb/IIIa receptors that are not blocked. Failure to agglutinate indicates either failure of the GPIIb/IIIa receptors to become activated and/or blockade of the GPIIb/IIIa receptors. The assay includes the ability to transfer blood to be tested from a collection container to an assay device without opening the collection container.

When the volume of blood needed to perform the test is greater than a few drops, a blood collection container such as a vacuum tube or syringe is used. The subsequent delivery of the sample into the assay requires the transfer of blood from the collection container to an assay device. The transfer increases the risk of both hazardous contact to the clinician and contamination of the sample. To minimize the risk to the danger to clinicians and laboratory technicians and to reduce the risk of sample contamination, sampling typically occurs directly from a sealed vacuum tube such that the tube cap does not have to be removed during the process. Exemplary tube manufacturers and their respective vacuum tube trade names include Becton Dickinson VACUTAINER®, Greiner Bio-One VACUETTE®, Sarstedt S-MONOVETTE® and Terumo VENOSAFE®.

Prior art discloses a wide variety of devices and methods for extracting fluid biological samples from a sealed vacuum tube. See, e.g., U.S. Pat. Nos. 5,279,796, 5,602,037, 5,888,826, 6,016,712, 6,817,256, 6,869,405 and 6,902,534. A typical extraction protocol involves piercing the septum or membrane of the vacuum tube with a needle, cannula or pipette and applying vacuum pressure to extract the fluid sample from the tube. In some cases, the vacuum tube is inverted by about 180° prior to aspiration, causing the sample fluid to move to the top of the sample tube. To further facilitate fluid transfer, some patents also disclose a second needle, cannula or pipette for equilibrating the pressure inside the sealed vacuum tube. See, e.g., U.S. Pat. Nos. 3,941,171, 4,296,786, 5,270,219, 5,380,486, 5,525,298, 5,837,203, 5,976,468, 6,271,043, 7,247,498, and U.S. Patent Pub. Nos. 2004/0228765 and 2007/0059204.

Based on the amount of fluid a vacuum tube is designed to hold, it may be classified as either a partial-draw tube or a full-draw tube. For example, the commonly owned U.S. Pat. No. 6,016,712, the contents of which is fully incorporated herein, discloses a first generation VERIFYNOW™ platelet function testing system (Accumetrics Inc., San Diego, Calif., U.S.A.) that was specifically designed for the use of sealed partial-draw vacuum tubes. To extract a fluid sample from the sealed partial-draw vacuum tube, a hubbed single needle is pressed onto and becomes a part of the Accumetrics consumable cartridge assembly.

The principal disadvantage of partial-draw vacuum tubes is that they tend to have higher fill-volume variability than do full-draw tubes. Sealed vacuum tubes usually contain a fixed volume of an anticoagulant, such as sodium-citrate, and any significant variation in the ratio of fluid-to-anticoagulant will have an adverse effect on the accuracy of testing results. Furthermore, since the main market suppliers of vacuum tubes have eliminated partial-draw tubes from their catalogs, the options available to consumers of partial-draw tubes have become severely limited. Thus, there is a compelling need in clinical diagnostics to convert assays to full-draw vacuum tube format.

However, using full-draw vacuum tubes for platelet function testing poses its own problem because a higher vacuum pressure is usually required to aspirate a fluid sample from a full-draw vacuum tube than is necessary for a partial-draw vacuum tube. The higher pressure results in damage to the cellular components of the fluid (e.g., hemolysis) and instigates platelet activation, which is the very thing that the diagnostic assay is trying to measure. Platelet activation induced by strong vacuum aspiration obfuscates the nominal patient platelet activation state and/or their response to drug therapy.

To date, no effective solution has been found that enables aspirating a fluid sample from a full-draw vacuum tube without damaging cells or activating platelets. Thus, there is a need to develop a reliable and inexpensive device for extracting a fluid sample from a closed container, particularly from a full-draw vacuum tube, without damaging cellular components or activating platelets.

### SUMMARY OF THE INVENTION

Accordingly, an object of the present invention is to provide a reliable and inexpensive device for extracting a fluid sample from a closed container, particularly from a full-draw vacuum tube, without damaging cellular components or activating platelets. Another object is to provide a method for extracting a fluid sample from a closed container, particularly from a full-draw vacuum tube, using the device of the present invention. Yet another object is to provide a kit for measuring platelet aggregation in a fluid sample that includes the device of the present invention.

One aspect of the present invention concerns a device for extracting a fluid sample from a closed container, such as a vacuum tube. The device comprises a housing hub having a first opening, a second opening, a venting port in fluid communication with the first opening, and an input port in fluid communication with the second opening. In some embodiments, the first opening is configured and adapted to accommodate a venting valve and a hydrophobic membrane. In some embodiments, the second opening is configured and adapted to mate with a tapered luer lock tip. The device further comprises a venting tip, such as a steel syringe needle, for maintaining a minimum pressure in the closed container. The venting tip has sharp and blunt ends, and the blunt end is engaged to the housing hub to establish fluid communication between the venting tip and the venting port. The device further comprises an input tip, such as another steel syringe needle, for extracting the fluid sample from the closed container. The input tip also has sharp and blunt ends, and the blunt end is engaged to the housing hub to establish fluid communication between the input tip and the input port. The device further comprises a venting valve, such as a duckbill check valve, positioned within the first opening for maintaining a desirable minimal pressure in the closed container. The venting valve permits a unidirectional air flow from outside the device to the closed container and prevents the fluid sample in the closed container from leaking out of the first opening. Finally, the device comprises a hydrophobic membrane positioned adjacent to the venting valve, which filters incoming air and prevents the fluid sample from leaking out of the device.

Another embodiment of the present invention is a method for extracting a fluid sample from a closed container. First, a fluid sample is provided in a closed container, such as a vacuum tube. A sample extracting device as described above is also provided. The closed container is pierced using the venting and input tips of the sample extracting device to establish fluid communication between the closed container and the sample extracting device. The sample extracting device is usually connected to a pressure altering device, such as a pump or a syringe, which is used to alter the pressure within the device to extract the fluid sample.

Yet another embodiment of the present invention is a kit for measuring platelet aggregation in a fluid sample. The kit comprises in a packaged combination a sample extracting device as described above and a reagent for measuring platelet aggregation. The kit may optionally comprise a sample collection container, such as a vacuum tube. The reagent for measuring platelet aggregation typically comprises a GPIIb/IIIa receptor ligand immobilized on a particle, an anticoagulant, and a buffer to maintain the pH and salt concentration of the fluid sample within a range suitable for platelet aggregation, as disclosed in the commonly owned U.S. Pat. No. 5,763,199, which is fully incorporated herein by reference.

### BRIEF DESCRIPTION OF THE DRAWINGS

FIG. 1 shows a perspective view of a hubbed dual cannula device according to the present invention.

FIG. 2 shows an alternative perspective view of a hubbed dual cannula device according to the present invention, with a transparent view of the housing hub.

FIG. 3A shows a sectional view of the device of FIG. 2 connected to a sealed vacuum tube and to an Accumetrics VERIFYNOW™ assay cartridge; FIG. 3B shows an enlarged sectional view of the same device.

### DETAILED DESCRIPTION OF THE INVENTION

Unless defined otherwise, all technical and scientific terms used herein have the same meaning as is commonly understood by one of ordinary skill in the art to which this invention belongs. All patents, patent applications (published or unpublished), and other publications referred to herein are incorporated by reference in their entireties. If a definition set forth in this section is contrary to or otherwise inconsistent with a definition set forth in the patents, applications, published applications and other publications that are incorporated herein by reference, the definition set forth in this section prevails over the definition that is incorporated herein by reference.

Citation of publications or documents is not intended as an admission that any of such publications or documents are pertinent prior art, nor does it constitute any admission as to the contents or date of these publications or documents.

As used herein, “a” or “an” means “at least one” or “one or more.”

As used herein, the term “fluid sample” refers to an isolated body fluid that may include cellular components and other particulate matter. The term encompasses both unprocessed fluid samples directly from a patient as well as fluid samples that have been pretreated and prepared in any convenient liquid medium, usually an aqueous medium (e.g., sodium citrate). The present invention has particular application to fluid samples that comprise platelets, such as, for example, whole blood, platelet-containing blood fractions such as plasma, platelet-rich plasma (PRP), and the like. Where platelet aggregation is to be measured, the fluid sample is typically whole blood subjected to less than about 50%, preferably less than about 20% dilution. The blood is preferably obtained from an extremity free of peripheral venous infusions, substantially in the absence of air.

As used herein, the term “engaged” refers to any mode of mechanical or physical attachment, interlocking, mating, binding, or coupling, such that members that are said to be “engaged” do not come apart or detach from one another without some positive effort, application of energy, or the like.

As used herein, the term “fluid communication” between two or more components refers to a connection, either direct or indirect (e.g., via a connector pipe communication), such that fluid can flow to and from those components communicating.

As used herein, the term “substantially equal” is defined as two measurable values being within about 30%, preferably within about 20%, more preferably within about 10%, and most preferably within about 5% or less of each other.

As used herein, the term “substantially parallel” is understood to mean “approximately parallel”, such as within about 30°, preferably within about 20°, more preferably within about 10°, and most preferably within about 5° or less of being perfectly parallel.

As used herein, the term “staggered in height” is intended to mean that the two needles are offset relative to one another, such that the sharp end of one needle extends farther from the housing hub than the sharp end of the other needle.

The term “non-coring needle” is a term of art and is used herein as such. It refers to a needle profile that permits the tip to be inserted into or through an object and/or removed from that object substantially without removing any material from the object. For example, in a typical embodiment, a tip having a substantially smooth outer surface and a tapered profile proximal to the end of the tip is inserted through an elastomeric septum that seals a container and is removed from the septum during a given fluid handling process substantially without removing any elastomeric material from the septum.

The term “duckbill check valve” is also a term of art and is used herein as such. A duckbill check valve is a type of check valve formed by two converging valve lips which meet at a slit wherein the lips are configured and adapted to move apart to open the slit to permit flow in a forward direction. The valve is usually made of a soft elastomeric material such that a positive seal is formed between the lips when the slit is closed to prevent leakage in a back flow direction.

As used herein, the term “partial-draw vacuum tube” refers to a vacuum tube that is designed to be partially filled with a fluid sample, whereas the term “full-draw vacuum tube” refers to a vacuum tube that is designed to be completely filled with a fluid sample. One common example of a partial-draw vacuum tube is the VACUETTE® tube (Greiner Bio-One, Monroe, N.C., U.S.A.), and one common example of a full-draw vacuum tube is the VACUTAINER® tube (Becton Dickinson, Franklin Lakes, N.J., U.S.A.). The vacuum tube preferably includes a small volume of a solution of sodium citrate generally in the range of about 35% sodium citrate having a volume in the range of about 0.05 to 0.5 ml.

As used herein, the term “single use device” refers to a device that is intended for just one use, i.e., on a single patient during a single procedure.

As discussed above, one aspect of the invention concerns a hubbed dual cannula device for extracting a fluid sample from a closed container. The closed container is usually a container in which the fluid sample to be processed is collected. The closed container may be in any form such as a syringe, a vacuum tube (e.g., a VACUTAINER® tube), a cuvette, a vial, a cartridge and the like. The vacuum tube may be either a partial-draw tube or a full-draw tube, more preferably a full-draw tube. Suitable materials for fabrication of the container are glass, plastic and the like. In general, any material may be used that does not react with, or otherwise cause detrimental effects on, the fluid sample or any solvents in which the fluid sample is dissolved. An appropriate element is included as part of the closed container for attachment to the hubbed dual cannula device in accordance with the present invention. For instance, the closed container preferably comprises an element capable of being pierced, such as a septum, membrane, and the like. The primary principle involved is that fluid sample can be transferred from the closed container to the present sample extracting device without opening the container.

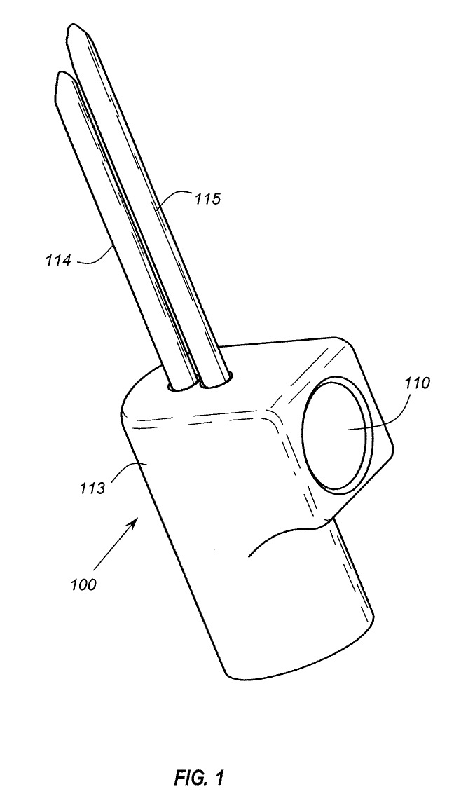

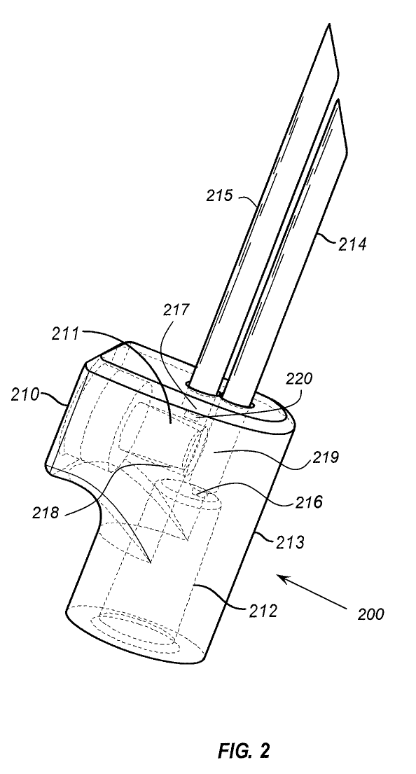

An exemplary embodiment of a hubbed dual cannula device according to the present invention is shown in FIG. 1 by way of illustration and not limitation. Device 100 is shown with a housing hub 113 comprising a hydrophobic membrane 110 and a venting valve (not shown). The housing hub is mated with a venting tip 115 and an input tip 114. Referring to FIG. 2, a fluid sample extracting device 200 according to the present invention comprises five basic components: a housing hub 213, two piercing tips 214 and 215 engaged to the housing hub 213, a venting valve 211 positioned within the housing hub 213 and a hydrophobic membrane 210 positioned adjacent to the venting valve 211.

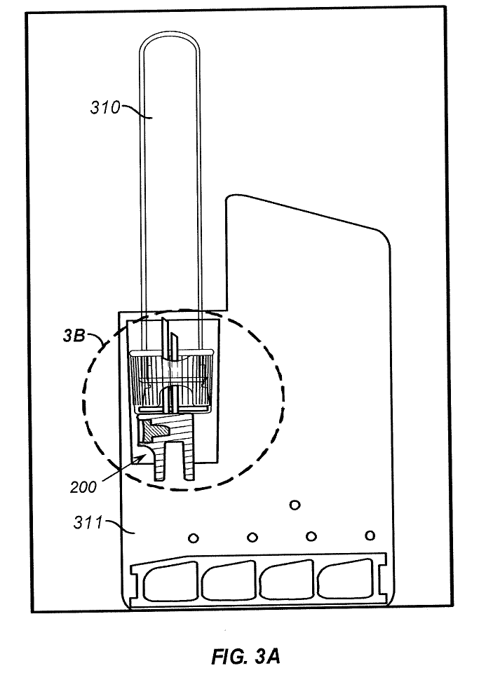

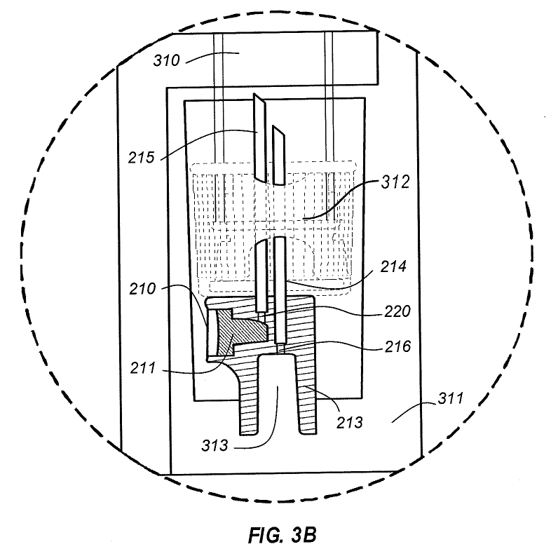

Referring now to FIGS. 2 and 3, the housing hub 213 is the main body of the assembly and, after assembly, contains or is engaged to all of the other device components. In order to accommodate the other components, the housing hub 213 comprises a first opening 218, which is configured and adapted to accommodate the venting valve 211 and the hydrophobic membrane 210, and a second opening 212, which is configured and adapted to accommodate a tapered luer lock tip 313. The first opening 218 is usually adapted for ready connection to the venting valve 211 by a mating means such as a compliant fitting, luer style fitting and the like. The housing hub 213 also comprises a venting port 220 for venting the sealed vacuum tube 310. The venting port 220 is in fluid communication with the first opening 218 and with a cylindrical passageway 217 adapted for mounting the venting tip 215. The housing hub further comprises an input port 216 that is in fluid communication with the second opening 212 and with a cylindrical passageway 219 adapted for mounting the input tip 214. The diameter of the venting and input ports is typically about 0.3 to 1.0 mm, preferably about 0.5 to 0.6 mm.

As explained above, the venting port 220 is in fluid communication with the first opening 218, which in turn is configured and adapted to accommodate the venting valve 211 for maintaining a desirable minimal pressure inside the sealed tube 310. The input port 216 provides for transferring fluid from the sealed tube 310 to a sample testing module 311, such as, for example, the Accumetrics VERIFYNOW™ assay cartridge, to which the sample extracting device 200 is usually attached via a tapered luer lock (212+313). A preferred means for facilitating such transfer is by decreasing the pressure inside the device. As explained above, the input port 216 is in fluid communication with the second opening 212 adapted for ready connection to the sample testing module 311, which may comprise a pressure altering device such as, for example, a syringe or a vacuum pump, as part of the testing module or be in fluid communication with a pressure altering device.

In some embodiments, at least one of the venting tip 215 and the input tip 214 comprises a syringe needle. The needle is preferably made of metal, such as stainless steel, similar to conventional syringe needles, although other suitably hard materials may be used as well. In some embodiments, the needle is about 16 to 26 gauge in size, more preferably at least about 21 gauge (e.g., Air-Tite Products Co., Inc., part No. N1812B). The dimensions of the needle are usually about 13 to 20 mm, preferably about 16 mm in length, about 0.6 to 1.5 mm, preferably about 0.8 mm, in outside diameter, and about 0.3 to 1.0 mm, preferably about 0.5 mm, in inside diameter. In some embodiments, the needle is a non-coring needle, preferably comprising a chisel tip. In some embodiments, the venting and input tips have substantially equal lengths. In some embodiments, the venting and input tips are substantially parallel to each other and their sharp ends are either staggered in height (as shown in FIGS. 1-3) or set to have substantially equal heights.

As explained above, the piercing tips 214 and 215 are engaged to the housing hub 213 by being mounted in cylindrical passageways 219 and 217, respectively. In some embodiments, the device may be manufactured with the piercing tips already secured in the housing hub. In some embodiments, the piercing tips may be secured in the housing hub prior to use. The cylindrical passageways 217 and 219 can be of any convenient length and diameter as long as they can hold the piercing tips to permit ready piercing of the sample container. The cylindrical passageway 219 is in fluid communication with the input port 216 to provide access of the fluid sample to the device. The device may further include a cover for the piercing tips to protect both the tips and the user.

The venting valve 211 permits air flow only in one direction, from outside the sample extracting device 200 to the sealed tube 310. The venting valve 211 also prevents the backflow of fluid from the sealed tube 310 to the first opening 218. Suitable venting valves include, for example, a check valve, such as a duckbill valve, a solenoid valve, a shuttle valve and so forth. In some embodiments, the venting valve 211 is a duckbill check valve. The duckbill check valve is a commercially-available component whose material, size, and design limits the free-flow of air until a minimum “cracking pressure” (i.e., the minimum pressure differential at which the valve will open) is achieved. This enables maintaining a desirable minimal pressure in the closed sample container.

The hydrophobic membrane 210 is positioned adjacent to the venting valve 211 and serves as a filter of incoming air that eventually makes its way into the sealed vacuum tube 310, and further serves as a failsafe secondary mechanism for preventing any backwash fluid from leaking out of the first opening 218. In some embodiments, the hydrophobic membrane comprises a material that is designed to swell and seal the first opening 218 in the presence of a fluid. Such materials may comprise, for example, porous polymers such as POREX XM-1374® (Porex Technologies, Inc., Fairburn, Ga., U.S.A.), GORE-TEX™ (W.L. Gore & Associates, Inc., Newark, Del., U.S.A.), and the like.

The hubbed dual cannula device may be fabricated by inserting the venting valve into the first opening of the housing hub, placing the hydrophobic membrane immediately behind the valve and cold-forming (i.e. compressing and deforming) a surrounding rim of plastic to achieve a fixed pre-load on the venting valve. The venting and input tips are then oriented, inserted into the cylindrical passageways of the housing hub, and fixed in place by means of a permanent adhesive. The housing hub may be fabricated by injection molding as a single piece, or alternatively it may be assembled from individual injection molded parts. The housing hub may be fabricated from a material that can withstand the temperatures employed in a processing of the sample. In general, any material may be used that does not react with, or otherwise cause detrimental effects on, the fluid sample or any solvents in which the fluid sample is dissolved or suspended. Suitable materials for the manufacture of the housing hub include thermoplastic materials, such as, for example, polystyrene, acrylonitrile butadiene styrene (ABS), polyamide, polypropylene, polyethylene terephthalate (PET), polycarbonate, polyvinyl chloride (PVC), and the like. In some embodiments, the present hubbed dual cannula device is a single use device that is discarded after each use.

Another embodiment of the present invention is a method for extracting a fluid sample from a closed container. Referring to FIGS. 2 and 3, a fluid sample is usually provided in a closed container, such as the vacuum tube 310. The hubbed dual cannula sample extracting device 200 as described above is also provided. The device 200 is preferably deployed by press-fitting it onto a suitable tapered luer lock tip 313 of a sample testing module 311, such as, for example, the Accumetrics VERIFYNOW™ assay cartridge, to establish fluid communication between the input needle 214 and the sample testing module. The sample module is plugged into a suitable clinical chemistry instrument, such as, for example, the Accumetrics VERIFYNOW™ System. The membrane or septum 312 of the vacuum tube 310 is then pierced by impaling the vacuum tube on the needles 215 and 214, as shown in FIG. 3B, to establish fluid communication between the vacuum tube 310 and the sample extracting device. After the septum of the vacuum tube has been pierced, the instrument uses internal pneumatics, first to extract the fluid sample from the vacuum tube and into the testing module's staging area where it is warmed, and then to pressurize the staging area to force the warmed sample from the staging area and into the testing module's mixing and detection chambers where the assay testing is actually performed.

The present method has a number of important advantages. First, by virtue of having an integrated venting valve, the pressure inside the closed container never falls below a certain pre-set threshold, which means that less force will be required to extract a fluid sample from the container. Therefore, the cellular components of the fluid sample will suffer less shear damage and platelets are less likely to become activated as a result of the transfer. Second, since the present hubbed dual cannula device is fully self-contained and inexpensive to manufacture, it is easily amenable to single use and does not require washing.

Another embodiment of the present invention is a kit for measuring platelet aggregation in a fluid sample that comprises in a packaged combination a sample extracting device as described above and a reagent for measuring platelet aggregation, as disclosed in U.S. Pat. Nos. 5,763,199, 5,854,005, 6,016,712, 7,205,115, U.S. Patent Pub. Nos. 2005/0031616 and 2006/0246528, and Ser. No. 12/114,498 (filed May 2, 2008), all of which are fully incorporated herein by reference. The kit preferably includes a lyophilized preparation comprising particles coated with a compound that promotes specific agglutination of platelets, a platelet activator and buffer. The lyophilized preparation may be present in a reaction container such as a cartridge used in the instrument of analysis. For the aforementioned Accumetrics VERIFYNOW™ System, the lyophilized preparation may be placed in the outer wells of the four-well cartridge used in the analyzer. The kit may also include a sample collection container and/or a device for carrying out the present method. In some embodiments, the sample collection container is a vacuum tube, preferably a partial-draw vacuum tube or a full-draw vacuum tube. The relative amounts of reagents may vary to provide for concentrations of the reagents in solution that substantially optimize the sensitivity of a determination.

As explained above, the kit typically comprises particles coated with a compound that can result in the specific agglutination of platelets, i.e., the agglutination of platelets by the specific interaction between a receptor on the platelets and the compound on the particles. Such compounds include, by way of illustration and not limitation, antibodies to a platelet receptor and GPIIb/IIIa receptor ligands, which may be a small organic molecule, polypeptide, protein, monoclonal antibody or nucleic acid that binds, complexes or interacts with GPIIb/IIIa receptors on the platelet surface. Platelet mediated aggregation of the particles results when the GPIIb/IIIa receptors on the surface of platelets bind, complex or otherwise interact with the GPIIb/IIIa receptor ligands on the particles. In some embodiments, GPIIb/IIIa ligands may include fibrinogen, monoclonal antibody 10E5 (Coller, et al., J. Clin. Invest. 1983, 72:325), monoclonal antibody c7E3 (The EPIC Investigators, N.E. J. Med. 1994, 330:956), von Willebrand factor, fibronectin, vitronectin and other ligands that have an arginine glycine-aspartic acid (RGD) sequence or other peptides or peptidomimetics that mimic this amino acid sequence (Cook, et al., Drugs of the Future 1994, 19:135). Other compounds of interest may include low molecular weight heparin or the like.

The kit may also include other reagents necessary for carrying out the assay of the present invention. For example, in some embodiments, the kit includes a sample vial, a buffer that maintains the pH and salt concentration of the fluid sample assessed within ranges suitable for platelet mediated agglutination of the solid surface and small polymeric beads coated with platelet GPIIb/IIIa receptor ligand. The buffer can be in solution, or can consist solely of the buffering composition and salts to which a known amount of water is added to give the desired buffer solution. Optionally, the kit can also comprise an anticoagulant. In some embodiments, the buffer is HEPES; the anticoagulant is citrate; a GPIIb/IIIa receptor ligand is fibrinogen; small polymeric beads are polyacrylonitrile or carboxylated polystyrene in which a peptide GPIIb/IIIa receptor ligand, such as fibrinogen, is covalently or passively bound to the bead surface.

Where appropriate, the reagents can be placed in an air-tight package in order to maintain the activity of any reagents. The package may be, for example, a bag, pouch, or the like fabricated from a material that is substantially non-permeable to moisture. Such materials include, for example, plastic, aluminum foil, and the like. The kit may further include an article for piercing a person's skin, disinfectant or sterilizing pads and so forth. The kit may also include calibrators and standards. Furthermore, the kit may also include one or more reagents for conducting an assay for platelet count. In some embodiments, the kit includes a single use sample extracting device as described above.

1. A device for extracting a fluid sample from a closed container, comprising:
(a) a housing hub comprising a first opening, a second opening, a venting port in fluid communication with the first opening, and an input port in fluid communication with the second opening,
(b) a venting tip having a sharp end and a blunt end, wherein the blunt end of the venting tip is engaged to the housing hub to establish fluid communication between the venting tip and the venting port;
(c) an input tip having a sharp end and a blunt end, wherein the blunt end of the input tip is engaged to the housing hub to establish fluid communication between the input tip and the input port;
(d) a venting valve positioned within the first opening, which valve permits a unidirectional air flow from outside the device to the closed container and prevents the fluid sample inside the closed container from leaking out of the first opening; and
(e) a hydrophobic membrane positioned adjacent to the venting valve, which membrane filters incoming air and prevents the fluid sample inside the closed container from leaking out of the first opening.

2. The device of claim 1, wherein the fluid sample is a whole blood sample.

3. The device of claim 1, wherein the fluid sample is a plasma sample.

4. The device of claim 3, wherein the plasma sample is a platelet rich plasma sample.

5. The device of claim 1, wherein the first opening is configured and adapted to accommodate the venting valve and the hydrophobic membrane.

6. The device of claim 1, wherein the second opening is configured and adapted to mate with a tapered luer lock tip.

7. The device of claim 1, wherein at least one of the venting and input tips comprises a syringe needle.

8. The device of claim 7, wherein the syringe needle is about 16 to 26 gauge.

9. The device of claim 7, wherein the syringe needle is a non-coring needle.

10. The device of claim 7, wherein the syringe needle comprises a chisel tip.

11. The device of claim 1, wherein the venting tip and the input tip have substantially equal lengths.

12. The device of claim 1, wherein the venting and input tips are substantially parallel to each other.

13. The device of claim 12, wherein the sharp ends of the venting and input tips are staggered in height or have substantially equal heights.

14. The device of claim 1, wherein the venting valve comprises a duckbill check valve.

15. The device of claim 1, wherein the hydrophobic membrane comprises a material that expands in the presence of a fluid.

16. The device of claim 1, wherein the housing hub comprises a thermoplastic material.

17. The device of claim 1, wherein the device is a single use device.

18. A method for extracting a fluid sample from a closed container, comprising the steps of:
(a) providing the sample extracting device of claim 1 and a fluid sample in a closed container;
(b) piercing the closed container using the sharp ends of the venting and input tips to establish fluid communication between the closed container and the sample extracting device;
(c) altering the pressure within the device to extract the fluid sample.

19. The method of claim 18, wherein the fluid sample is a whole blood sample.

20. The method of claim 18, wherein the fluid sample is a plasma sample.

21. The method of claim 20, wherein the plasma sample is a platelet rich plasma sample.

22. The method of claim 18, wherein the closed container comprises a vacuum tube.

23. The method of claim 22, wherein the vacuum tube is selected from a full-draw vacuum tube and a partial-draw vacuum tube.

24. A kit for measuring platelet aggregation in a fluid sample, the kit comprising the sample extracting device of claim 1 and a reagent for measuring platelet aggregation.

25. The kit of claim 24, further comprising a sample collection container.

26. The kit of claim 25, wherein the sample collection container is a vacuum tube.

27. The kit of claim 26, wherein the vacuum tube is selected from a full-draw vacuum tube and a partial-draw vacuum tube.

28. The kit of claim 24, wherein the reagent for measuring platelet aggregation comprises a GPIIb/IIIa receptor ligand immobilized on a particle.

29. The kit of claim 28, further comprising an anticoagulant and a buffer to maintain the pH and salt concentration of the fluid sample within a range suitable for platelet aggregation.

30. The kit of claim 28, wherein the GPIIb/IIIa receptor ligand comprises a substance selected from the group consisting of fibrinogen, monoclonal antibody 10E5, monoclonal antibody c7E3, von Willebrand factor, fibronectin, vitronectin, a ligand having an arginine glycine-aspartic acid (RGD) sequence and a peptide or a peptidomimetic mimicking RGD sequence.

31. The kit of claim 28, wherein the GPIIb/IIIa receptor ligand comprises fibrinogen.

32. The kit of claim 24, wherein the sample extracting device is a single use device.
